(19) United States
(12) Patent Application Publication
(10) Pub. No.: US 2007/0208285 A1
(43) Pub. Date: Sep. 6, 2007

(54) Itchblocker air cast system

(76) Inventor: Tyrone Blocker, Oregon City, OR (US)

Correspondence Address: LAW OFFICE OF KAREN DANA OSTER, LLC, PMB 506, 16869 SW 65TH AVENUE, LAKE OSWEGO, OR, 97035, US

(21) Appl. No.: 11/367,502

(22) Filed: Mar. 2, 2006

(51) Int. Cl. A61F 5/00

(52) U.S. Cl. 602/2

(57) ABSTRACT

A small light weight compressor/air pump(1) powered by two interchangeable rechargeable battery cells one powering the unit while the other is being recharged (2). The pump nozzle will be attached to a air hose approximately ½ to ⅜ inch in diameter. The pump outlet hole should be between ¼ and ½ inch (6). Air flow i estimate at about 2 cubic feet per minute. 1 cu.ft. (0.028 Cu.m. ) minimum. (5). The hose will attach to a plastic bag that will be inserted into the cast between the stockinette and cotton padding. The plastic bag will be vented with air passages at two to three inch intervals to assure air circulation throughout the casted area.

### CROSS-REFERENCE TO RELATED APPLICATIONS

“This application claims the benefit of patent application Ser. No. 10/821,115.

### FEDERALLY SPONSORED RESEARCH

Not Applicable

### SEQUENCE LISTING OF PROGRAM

Not Applicable

### BACKGROUND OF THE INVENTION—FIELD OF INVENTION

This invention relates to the casting of orthopedic patients, specifically Cutaneous Complications inside the cast.

### BACKGROUND OF THE INVENTION

Orthopedic doctors perform all castings of patients suffering from fractures within their bodies.

There is no known treatment for the prevention of cutaneous complications, which occur inside patients cast. However, Universaly the majority of Orthopaedic Surgeans, write in their guidelines for Orthopaedic Technology, Cast Care Guides, and various Sports Articles, how traditional cast construction such as plaster of paris or fiberglass, often results in Cutaneous Complications, “including Macerations, Ulcerations, Infections, Burns, Blisters, Rashes, and Allergic contact Dermatitis.” Orthopaedic Surgeons and Clinical Professors write that, patients often reports that such cast itch, smell, and are difficult to keep dry. Further, they write that, “plaster of paris cast break down if patients get them wet. Although fiberglass cast with stockinette and cast padding can withstand moisture, they must be dried, perhaps with a hair dryer to minimize cutaneous complications. Maceration, odor, and itching frequently develope when damp areas remain beneath the fiberglass cast.”

It is universally excepted throughout the world that cool air is the solution to either preventing or minimizing cutaneous complications beneath the cast, but until Tyrone's new method of inserting an attachment inside the cast, the only other method was to use a hair dryer. Tyrone believes that the hair dryer is not only inconvenient to use for that purpose, but is not designed to pump cool air, and has a great potential for danger to the patient especially children. Furthermore, because the cast has to be fitted tightly against the patients fracture to stablelize the broken bone, the hair-dryer would not be capable of pushing enough air through most cast.

Accordingly, several objects and advantages of the present invention are:

(a) to provide a treatment for the prevention of cutaneous complications, which occur inside patients cast.

(b) to provide a treatment that would allow the patients fracture to heal in a more timely fashion while greatly reducing the risk of permanent injury due to cutaneous complications within the cast.

(c) to provide a treatment which will reduce inflated medical cost to the patient due to return visits to redress cutaneous complications within the cast.

(d) to provide a treatment that will keep the cast from breaking apart due to moisture build-up inside the cast.

(e) to provide a treatment that will minimize cutaneous complications including macerations, ulcerations, and Infections.

(f) to provide a treatment that will minimize or prevent, burns, blisters, rashes, and allergic contact dermatitis.

(g) to provide a treatment that will minimize or prevent discomfort, itching, and odor within the cast.

Further objects and advantages are to provide a device which can be easily and conveniently used, which can be made light weight and small in size, which can be applied to the patient without any potential harm to the patient and can be made to be hand held or hooked on the waist of the patient making it convenient for patients to move around. Still further objects and advantages will become apparent from a consideration of the ensuing description and drawings.

### SUMMARY

The itchblocker air cast system is a method for minimizing or preventing cutaneous complications within a cast without potential harm to the patient comprising pumping cool temperature air through the cast to prevent heat, moisture, and bacteria build-up inside, by use of a compressor/air pump with tubing connections and air inserts that are specifically designed to be placed inside the patients cast.

### DESCRIPTION

1. Light Weight compressor/air pump. 31 lbs(1.36 kg)max.

2. Power preferably uses two interchangeable rechargeable battery cells. One would power the unit while the other is being recharged. The unit or battery cell should indicate when power levels are low and charging is required. The charger should have lights to indicate operation and charging is complete.

3. Switches the pump only need a water proof on-off switch. It is desired to have the on mode be timed by electronic circuit.

4. Noise & Vibration quite and low vibration is important A damp and isolated double housing is recommended.

5. Air Flow i estimate the flow at about 2 cubic feet per minute. 1 cu.ft. (0.028 cu.m.) minimum.

6. Nozzle the pump will be attached to an air hose approximately ½ to ⅜ inch in diameter. The pump outlet hole should be between ¼ and ½ inch.

7. Dimensions the smaller, the better.

Attachment

The insert is made up from a sheet of plastic connected to a light flexible spring and plastic spout sandwiched inside a thin durable plastic bag known as seal a meal, which is vented with specially designed air passages. The sheet of plastic and spring inside the bag keeps the bag in place and assures continuous maximum air flow.

The insert is designed to enter within the cast material between the stockinette and cast padding.

Accessories

The product will be designed to attach to a belt and hand held. This feature does not have to be part of the pump.

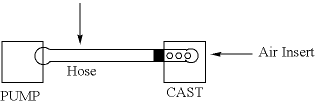

### CONCLUSION, RAMIFICATION, and SCOPE

Accordingly, the reader will see that the itchblocker air cast system is a method for minimizing or preventing cutaneous complications beneath a cast, which includes macerations, ulcerations, infections, burns, blisters, rashes, and allergic contact dermatitis. Comprising pumping cool temperature air through the patients casted area to prevent heat, moisture and bacteria build up, which causes the cutaneous complications by use of a compressor/air pump and inserts that are specifically designed to fit inside the cast.

Furthermore, the method will allow the patients fracture to heal in a more timely fashion while greatly reducing the risk of permanent injury due to cutaneous complications beneath the cast. In addition, the itchblocker air cast system, has the objects and advantages in that:
it provides a treatment which will reduce inflated medical cost to the patient due to return visits to redress cutaneous complications beneath the cast.
it provides a treatment that will keep the cast from breaking apart due to moisture build-up inside the cast, giving the cast longer life.
it provides a treatment that will minimize or prevent cutaneous complications, including macerations, ulcerations, and infections.
it provides a treatment that will minimize or prevent burns, blisters rashes, and allergic contact dermatitis.
it provides a treatment that will minimize or prevent discomfort, itching, and odor beneath the cast.
Although the description above contains many specificities, these should not be construed as limiting the scope of the method invention, but as merely providing illustrations of some of the presently preferred embodiments of this method invention. For example, different blowers, tubing, or inserts can be used as well as different air pressures, temperatures, and power supplies units.

Thus the scope of the method invention should be determined by the appended claims and their legal equivalents, rather than by the examples given.

I claim:

1. A method for minimizing or preventing cutaneous complications inside a cast comprising pumping cool temperature air through the casted area to prevent heat, moisture, and bacteria build-up inside, by using a compressor/air pump and air inserts, which is specifically designed to be placed inside of the cast.
